(19) United States
(12) Patent Application Publication
(10) Pub. No.: US 2026/0041736 A1
(43) Pub. Date: Feb. 12, 2026

(54) COMPOSITIONS AND METHODS FOR FACILITATING HEART AND LUNG REPAIR

(71) Applicants: William Marsh Rice University, Houston, TX (US); Baylor College of Medicine, Houston, TX (US)

(72) Inventors: Omid VEISEH, Houston, TX (US); Amanda NASH, Houston, TX (US); Samira AGHLARA-FOTOVAT, Houston, TX (US); Aarthi PUGAZENTHI, Houston, TX (US); Ravi GHANTA, Houston, TX (US); Miguel A. MENDEZ SOSA, Houston, TX (US)

(21) Appl. No.: 19/100,392

(22) PCT Filed: Aug. 2, 2023
(86) PCT No.: PCT/US2023/071514

§ 371 Date: Jan. 31, 2025

Related U.S. Application Data
(60) Provisional application No. 63/394,481, filed on Aug. 2, 2022.

(51) Int. Cl. A61K 38/20; A61K 9/00; A61K 35/36; A61P 9/10

(52) U.S. Cl. CPC A61K 38/2066; A61K 9/0024; A61K 35/36; A61P 9/10

(57) ABSTRACT

The present disclosure relates to implantable constructs designed to deliver therapeutic reagents to a subject for the induction of tissue regeneration in damaged or disease lung and heart tissues. In certain aspects, the constructs are designed to degrade over time or upon a particular signal, thereby providing control of the length of time the therapeutic agent is delivered to the subject.

### PRIORITY CLAIM

This application claims benefit of priority to U.S. Provisional Application Ser. No. 63/394,481, filed Aug. 2, 2022, the entire contents of which are hereby incorporated by reference.

### STATEMENT REGARDING FEDERALLY SPONSORED RESEARCH

This invention was made with government support under Grant No. R01DK120459, awarded by the National Institutes of Health and Grant No. 1842494, awarded by the National Science Foundation. The government has certain rights in the invention.

### BACKGROUND

### I. Field

The present disclosure relates to the fields of biology, medicine, bioengineering and medicals devices. More particularly, it relates to the development and use of implantable constructs designed to deliver therapeutic reagents to a subject and protect the subject from an unwanted immune response. In particular, the implantable constructs are designed to deliver cytokines and other agents to induce tissue repair after heart of long damage.

### II. Related Art

Advances in biomedical research have led to methods for localized and targeted therapies for the treatment of diseases. However, in many instances, the percentage of patients responsive to these approaches remain modest (Park et al., Sci. Transl. Med. 10(433) 2018).

One approach involves the use of implantable devices to deliver therapeutic agents. A fundamental barrier to successful device-based therapies is the lack of biocompatible transplantation devices. Thus, a critical medical need exists to develop biomaterials that overcome key challenges in this field.

### SUMMARY

The present disclosure provides, at least in part, a method of treating tissue damage and/or inducing tissue regeneration in a subject comprising providing an encapsulated engineered cell expressing a cytokine; and administering to said subject said encapsulated cell. The engineered cell may be any type of cell, for example, a Chinese hamster ovary (CHO) cell, retinal pigment epithelial (ARPE-19) cell, human mammary epithelial (MCF-10a and MCF-7) cell, human embryonic kidney (HEK) cell, a mesenchymal stem cell (MSC), human umbilical vein endothelial cell (HUVEC), NIH/3T3 cell, BJ fibroblast, or human renal mix epithelial cell (HREC), such as wherein said cell is engineered for regulatable expression of said cytokine. In an embodiment, the cytokine may be an anti-inflammatory or pro-inflammatory cytokine. In an embodiment, the cytokine is IL-10. The material that encapsulates said engineered cell may be degradable. The material that encapsulates said engineered cell may be a naturally occurring or synthetic polymer. For example, material may be a polysaccharide, e.g., alginate. The material that encapsulates said engineered cell may further comprise a compound, for example, a compound comprising a triazole moiety The tissue may be heart or lung tissue, such as heart tissue damaged by ischemia, such as due to coronary heart disease and/or myocardial infarction. The method lung tissue may have been damaged by infection, such as by viral infection. Administration may comprise administration directly to damaged tissue.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. A schematic illustration of the platform depicting development of engineered cells for production of various anti-inflammatory cytokines. These engineered cells are subsequently encapsulated within alginate hydrogels, termed “cytokine factories”.

FIG. 2. Schematic illustration of the IL-10 and IL-1 signaling pathways.

FIG. 3. The anti-inflammatory cytokine factory will be encapsulated within a biocompatible polymer in order to preserve cell viability in vivo. Retinal pigment epithelial cells engineered to produce human IL-10 were encapsulated within such a hydrogel and evaluated for sustained viability and function. Live dead staining of encapsulated RPE-hIL10 cells demonstrates no significant loss of viability following encapsulation. To evaluate the change in cytokine production following encapsulation, cells were plated in 2D, or encapsulated and incubated for 24 hours. supernatant was collected and analyzed via ELISA for hIL10 production. Results show no significant decrease in cytokine production following encapsulation.

FIG. 4. One major advancement of this technology over recombinant cytokine administration is the ability to localize the therapy to a target tissue. To demonstrate the ability of RPE-IL10 capsules to remain viable locally for an extended duration of time, encapsulated cells engineered with a firefly luciferase reporter were implanted in the pericardial sac of Sprague Dawley rats. An in vivo imaging system (IVIS) was used to demonstrate cell localization over time. Results demonstrate that 3 weeks post implant, cells maintain luminescent signal and are localized to the left pleural cavity (n=3, 0.5 mL capsules/n). To demonstrate the ability of the inventors' cytokine factories to achieve high local concentrations without significant effect on systemic cytokine concentrations, the inventors used ELISA to quantify hIL10 in pleural fluid compared to plasma. Results show that pleural fluid IL-10 is significantly greater (˜30,000×) than in the plasma highlighting the safety profile of this platform.

FIGS. 5A-C. The inventors have previously demonstrated the therapeutic mechanism of IL-10 Treatment. Generally, IL-10 capsules administered following acute MI increased M2-like macrophages and regulatory T cells. Immune mapping of nine rat hearts by CyTOF with 40 protein markers read 27,874 immune cells. Eight immunophenotypes in CD45+ immune cells were generated based on representative phenotypic surface markers. Compared to wild type rat hearts, MI and its treatment with IL-10 led to dramatic alteration of macrophages and CD4 T cell populations. Furthermore, IL-10 treatment led to a significant increase of CD163(+) macrophages (M2-like macrophage) and regulatory T cells. In contrast, CD163(−)CD206(−) macrophages (M1-like macrophage) were significantly decreased after IL-10 treatment.

FIG. 6. Functional CyTOF has demonstrated cytokine production in key cell types following IL-10 capsule administration. Activated Tregs expressing Ki67 are releasing IL-10 and IL-17 to induce macrophage differentiation, which are measured after Golgistop protein transport inhibitor treatment for 12 hours.

FIGS. 7A-B. IL-10 treatment led to significant increases in IL-10 in infarcted hearts compared to non-treated infarcts. Higher intra-cellular IL-10 was measured in myofibroblasts, fibroblasts, macrophages, and regulatory T-cells. MMI=mean mass intensities.

FIG. 8. The inventors have previously demonstrated the potential of this platform for therapeutic applications in the pleural cavity. Specifically, encapsulation of mesenchymal stem cells demonstrated significant improvements in myocardial function following acute MI. Prior to efficacy experiments, production of various MSC specific paracrine factors were evaluated via ELISA. Similar to what is described previously, cells were plated in 2D and production of factors was compared to that of encapsulated MSCs. Results demonstrated no significant reduction of paracrine factor production following encapsulation. After validation of sustained MSC factor production post encapsulation, MSCs were implanted in the pericardial space following acute MI via left anterior descending artery ligation. Encapsulated MSCs were compared to MSCs injected directly into the pericardium and a control (blank) capsule group (n=5). Results demonstrated that left ventricular ejection fraction (LVEF) MSC injection (*; p<0.05) and MSC capsules (**; p<0.05) compared to post-infarct. Furthermore, percent change in EF for individual subjects showed greatest improvement in MSC capsule group compared to MSC injection group (41% vs 16%; p<0.05). Finally, analysis of Masson's Trichrome-stained myocardial sections 28 days after MI and treatment demonstrated a significant reduction in fibrotic tissue following treatment with encapsulated MSCs. *p<0.05, **p<0.01 FIG. 9. CD4+ subsets regulate ECM profile and angiogenic chemokine secretion of fibroblasts. (Left) Fibroblasts (P3) treated with condition media from activated lymphocytes demonstrating upregulation of ECM turnover genes (Collagen3a1, MMP8, and MMP9) by CD4+(non-Treg or CD4+CD25+ Treg) (n=3). (Right) SDF1 levels in adult fibroblasts (AFB) with or without lenti-IL10 transduction (4 days post transduction). *p<0.05.

FIG. 10. RPE-IL10 improves LV function after MI. LV EF before and after coronary ligation and treatment with 20 capsules of naïve RPE or RPE-IL10 after 28 days (naïve RPE vs RPE-IL10; n=3/group; p<0.05). Representative (Masson's trichrome) sections of myocardium 28 days after infarct. Naïve RPE capsules (left) and RPE-IL10 (right), demonstrating marked decrease (13±3% vs 23±4%, p<0.05) in extent of fibrosis. * p<0.05.

FIGS. 11A-E. Development and validation of encapsulated anti-inflammatory cytokine factories in vitro and in vivo. (FIGS. 11A-B) Encapsulated cells engineered to produce Rat interleukin 1 receptor antagonist (RIL1Ra) and Rat interleukin 10 (RIL10) as well as Human interleukin 1 receptor antagonist (HIL1Ra) and Human interleukin 10 (HIL10) exhibit sustained high concentrations of cytokine production. (FIG. 11C) Viability assessment of encapsulated cells through Live/Dead staining reveals over 90% cell viability. (FIGS. 11D-E) Implantation of encapsulated Rat IL1Ra and IL10 cells in the pleural space demonstrates elevated local cytokine concentrations, with systemic circulation concentrations reduced by up to 100× at 1-, 3-, 7-, and 28-days post-implant. (FIG. 11F) Darkfield microscopy images of explanted capsules showcase the fibrosis-mitigating capability of anti-inflammatory cytokine-producing capsules for a duration of up to 28 days.

FIGS. 12A-B. Histological scoring of lungs with or without therapeutic capsule treatment. (FIG. 12A) Histological scores from n=5 rats at 1-, 7-, 14-, and 28-days post treatment with LPS alone or LPS plus therapeutic IL-10 and IL1Ra capsules. (FIG. 12B) Representative histological images of lung sections illustrating infiltration of different immune cells over time, and improved lung histology in treatment group by day 14.

FIGS. 13A-E. (FIG. 13A) Successfully transfected RPE cells transiently express mCherry and increase in number throughout antibiotic selection. Pre-selection (left), 5 days post-selection (right). (FIG. 13B) Cells treated with DOX for 48 hours produce significantly greater concentrations of anti-inflammatory cytokines compared to untreated cells (n=3). (FIG. 13C) DOX exposure time shares and exponential relationship with anti-inflammatory cytokine X production (n=3). (FIG. 13D) Representative images of capsules synthesized using various voltages demonstrate that increasing applied voltage results in decreasing capsule diameter. (FIG. 13E) Quantification of various capsule diameters, showing good reproducibility within groups (n=10).

### DETAILED DESCRIPTION

The present disclosure features implantable constructs for delivery of cells that express one or more therapeutic agents to a subject in a controlled release manner, and related methods of use thereof. The implantable constructs disclosed herein may be formulated into different morphologies (e.g., spheres, rods, tubes), and may be prepared using a variety of materials.

Each of these embodiments will be described below in more detail.

### A. DEFINITIONS

“Antigenic agent,” as used herein, is a substance which induces, activates, or evokes an immune response, e.g., in a subject.

“Cell,” as used herein, refers to an individual cell. In an embodiment, a cell is a primary cell or is derived from a cell culture. In an embodiment, a cell is a stem cell or is derived from a stem cell. A cell may be xenogeneic, autologous, or allogeneic. In an embodiment, a cell is be engineered (e.g., genetically engineered) or is not engineered (e.g., not genetically engineered).

“Degradable,” as used herein, refers to a structure which upon modulation, e.g., cleavage, decreases the ability of the implantable construct to impede contact of a host immune effector with the engineered cell. For example, the degradable entity can comprise a site which is cleavable by an enzyme, e.g., an endogenous host enzyme, or an administered enzyme. Typically, the degradable entity mediates a physical property of the encapsulated engineered cell, e.g., the thickness, degree of cross-linking, or permeability, which impedes passage of a host agent (e.g., a host immune component, e.g., a host immune cell).

“Prevention,” “prevent,” and “preventing” as used herein refers to a treatment that comprises administering or applying a therapy, e.g., administering an implantable construct (e.g., as described herein) comprising a therapeutic agent (e.g., a therapeutic agent described herein) prior to the onset of a disease or condition in order to preclude the physical manifestation of said disease or condition. In some embodiments, “prevention,” “prevent,” and “preventing” require that signs or symptoms of the disease or condition have not yet developed or have not yet been observed. In some embodiments, treatment comprises prevention and in other embodiments it does not.

“Subject,” as used herein, refers to the recipient of the implantable construct described herein. The subject may include a human and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development (e.g., a male or female of any age group, e.g., a pediatric subject (e.g., infant, child, adolescent) or adult subject (e.g., young adult, middle-aged adult, or senior adult). A non-human animal may be a transgenic animal.

“Treatment,” “treat,” and “treating,” as used herein, refer to reversing, alleviating, delaying the onset of, or inhibiting the progress of one or more of a symptom, manifestation, or underlying cause of a disease or condition. (e.g., as described herein), e.g., by administering or applying a therapy, e.g., administering an implantable construct comprising a therapeutic agent (e.g., a therapeutic agent described herein). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease or condition. In an embodiment, treating comprises reducing, reversing, alleviating, reducing, or delaying the onset of, an underlying cause of a disease or condition. In some embodiments, “treatment,” “treat,” and “treating” require that signs or symptoms of the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

### B. IMPLANTABLE CONSTRUCTS

An implantable construct described herein comprises a material that reduces or inhibits a reaction (e.g., such as an immunomodulatory reaction) with an engineered cell disposed within. For example, an implantable construct may comprise a material that shields the engineered cell from exposure to the surrounding milieu, such as host tissue, host cells, or host cell products. In an embodiment, an implantable construct minimizes the effect of a host response (e.g., an immune response) directed at an engineered cell disposed within, e.g., as compared with a similar cell that is not disposed within an implantable construct.

The implanted construct make take any shape. The surface may be flat surface or a curved surface, and can take a variety of more complex forms such a sphere, a tube (e.g., inside or outside of the tube), a bead, a rod, a wire, or even more complex 3-D structures such as medical devices.

The implantable construct may comprise a permeable, semi-permeable, or impermeable material to, for example, control the flow of solution in and out of the implantable construct and/or adopt the shape or size of its surroundings. For example, the material may be permeable or semi-permeable to allow free passage of small molecules, such as nutrients and waste products, in and out of the construct. In addition, the material may be permeable or semi-permeable to allow the transport of an cytokine, out of the implantable construct. Exemplary materials include polymers, metals, ceramics, and combinations thereof.

In an embodiment, the implantable construct comprises a polymer (e.g., a naturally occurring polymer or a synthetic polymer). For example, a polymer may comprise polystyrene, polyester, polycarbonate, polyethylene, polypropylene, polyfluorocarbon, nylon, polyacetylene, polyvinyl chloride (PVC), polyolefin, polyurethane, polyacrylate, polymethacrylate, polyacrylamide, polymethacrylamide, polymethyl methacrylate, poly(2-hydroxyethyl methacrylate), polysiloxane, polydimethylsiloxane (PDMS), polyhydroxyalkanoate, PEEK®, polytetrafluoroethylene, polyethylene glycol, polysulfone, polyacrylonitrile, collagen, cellulose, cellulosic polymers, polysaccharides, polyglycolic acid, poly(L-lactic acid) (PLLA), poly(lactic glycolic acid) (PLGA), polydioxanone (PDA), poly(lactic acid), hyaluronic acid, agarose, alginate, chitosan, or a blend or copolymer thereof. In an embodiment, the implantable construct comprises a polysaccharide (e.g., alginate, cellulose, hyaluronic acid, or chitosan). In an embodiment, the implantable construct comprises hyaluronic acid. In an embodiment, the implantable construct comprises alginate. In some embodiments, the average molecular weight of the polymer is from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). The implantable construct may comprise at least 0.5%, 1%, 2%, 3%, 4%, 5%, 7.5%, 10%, 15%, 20%, 30%, 40%, 50%, 60%, 70%, 80% or more of a polymer, e.g., a polymer described herein.

In an embodiment, the implantable construct comprises a polysaccharide, e.g., hyaluronic acid or an alginate. Alginate is a naturally occurring polymer comprising β-(1-4)-linked mannuronic acid and guluronic acid residues, and as a result of its high density of negatively charged carboxylates, may be cross-linked with certain cations to form a larger structure, such as a hydrogel. Alginate polymers described herein may have an average molecular weight from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). In an embodiment, the implantable construct comprises at least 0.5%, 1%, 2%, 3%, 4%, 5%, 7.5%, 10%, 15%, 20%, 30%, 40%, 50%, 60%, 70%, 80% or more of an alginate polymer. In an embodiment, the alginate is an ultrapure alginate (e.g., SLG20 alginate).

In an embodiment, the implantable construct comprises a metal or a metallic alloy. Exemplary metals or metallic alloys include titanium (e.g., nitinol, nickel titanium alloys, thermo-memory alloy materials), platinum, platinum group alloys, stainless steel, tantalum, palladium, zirconium, niobium, molybdenum, nickel-chrome, cobalt, tantalum, chromium molybdenum alloys, nickel-titanium alloys, and cobalt chromium alloys. In an embodiment, the implantable construct comprises stainless steel grade. The implantable construct may comprise at least 0.5%, 1%, 2%, 3%, 4%, 5%, 7.5%, 10%, 15%, 20%, 30%, 40%, 50%, 60%, 70%, 80% or more of a metal or metallic alloy, e.g., a metal or metallic alloy described herein.

In an embodiment, the implantable construct comprises a ceramic. Exemplary ceramics include carbide, nitride, silica, or oxide materials (e.g., titanium oxides, hafnium oxides, iridium oxides, chromium oxides, aluminum oxides, and zirconium oxides). The implantable construct may comprise at least 0.5%, 1%, 2%, 3%, 4%, 5%, 7.5%, 10%, 15%, 20%, 30%, 40%, 50%, 60%, 70%, 80% or more of a ceramic, e.g., a ceramic described herein.

In an embodiment, the implantable construct may comprise glass. The implantable construct may comprise at least 0.5%, 1%, 2%, 3%, 4%, 5%, 7.5%, 10%, 15%, 20%, 30%, 40%, 50%, 60%, 70%, 80% or more glass.

A material within an implantable construct may be further modified, for example, with a chemical modification. For example, a material may be coated or derivatized with a chemical modification that provides a specific feature, such as an immunomodulatory or antifibrotic feature. Exemplary chemical modifications include small molecules, peptides, proteins, nucleic acids, lipids, or oligosaccharides. The implantable construct may comprise at least 0.5%, 1%, 2%, 3% 4%, 5%, 7.5%, 10%, 15%, 20%, 30%, 40%, 50%, 60%, 70%, 80% or more of a material that is chemically modified, e.g., with a chemical modification described herein.

In some embodiments, the material is chemically modified with a specific density of modifications. The specific density of chemical modifications may be described as the average number of attached chemical modifications per given area. For example, the density of a chemical modification on a material in, on, or within an implantable construct described herein may be 0.01, 0.1, 0.5, 1, 5, 10, 15, 20, 50, 75, 100, 200, 400, 500, 750, 1,000, 2,500, or 5,000 chemical modifications per square μm or square mm.

In an embodiment, the chemical modification of a material may include a linker or other attachment moiety. These linkers may include a cross-linker, an amine-containing linker, an ester-containing linker, a photolabile linker, a peptide-containing linker, a disulfide-containing linker, an amide-containing linker, a phosphoryl-containing linker, or a combination thereof. A linker may be labile (e.g., hydrolysable). Exemplary linkers or other attachment moieties is summarized in Bioconjugate Techniques (3^{rd} ed, Greg T. Hermanson, Waltham, MA: Elsevier, Inc, 2013), which is incorporated herein by reference in its entirety.

### C. TRIAZOLES

In one aspect, the surface is made of a material that is itself immune evoking. In other aspect, the surface is coated with a distinct material that is either immune evoking or immune evasive. The inventors have developed a library of such compounds represented by the following formulae:
a compound of the formula:

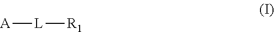

wherein:
   A is a polymer
   L is a linker of the formula:

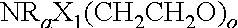

wherein:
   Rₐ is hydrogen, alkyl_{(C≤6)}, or substituted alkyl_{(C≤6)};
   o is 2, 3, 4, or 5; and
   X₁ is alkanediyl_{(C≤8)} or substituted alkanediyl_{(C≤8)};
or a linker of the formula:

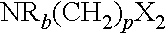

wherein:
   R_{b} is hydrogen, alkyl_{(C≤6)}, or substituted alkyl_{(C≤6)};
   p is 1, 2, or 3; and
   X₂ is arenediyl_{(C≤12)} or substituted arenediyl_{(C≤12)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynye_{(C≤6)} substituted aryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl; 4-alkoxy_{(C≤6)} substituted aryl_{(C≤12)}; or a group of the formula:

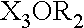

wherein:
   X₃ is alkanediyl_{(C≤8)} or substituted alkanediyl_{(C≤8)};
   R₂ is aryl_{(C≤12)} or substituted aryl_{(C≤12)};
or a pharmaceutically acceptable salt thereof.

The compound may be further defined as:

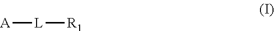

wherein:
   A is a polymer
   L is a linker of the formula:

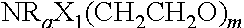

wherein:
   Rₐ is hydrogen, alkyl_{(C≤6)}, or substituted alkyl_{(C≤6)};
   m is 2, 3, 4, or 5; and
   X₁ is alkanediyl_{(C≤8)} or substituted alkanediyl_{(C≤8)};
R₁ is a cycloalkyl_{(C≤12)}; haloaryl_{(C≤12)}; S containing heteroaryl_{(C≤12)}; substituted S-containing heteroaryl_{(C≤12)}; alkyl_{(C≤6)}, haloalkyl_{(C≤6)}, alkenyl_{(C≤6)}, or alkynye_{(C≤6)} substituted aryl_{(C≤12)}; 3-aminophenyl; 4-alkoxy_{(C≤6)} substituted aryl_{(C≤12)}; or a group of the formula:

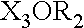

wherein:
   X₃ is alkanediyl_{(C≤8)} or substituted alkanediyl_{(C≤8)};
   R₂ is aryl_{(C≤12)} or substituted aryl_{(C≤12)};
or a pharmaceutically acceptable salt thereof.

The compound may be further defined as:

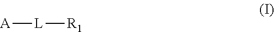

wherein:
   A is a polymer
   L is a linker of the formula:

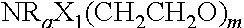

wherein:
   Rₐ is hydrogen, alkyl_{(C≤6)}, or substituted alkyl_{(C≤6)};
   m is 2, 3, 4, or 5; and
   X₁ is alkanediyl_{(C≤8)} or substituted alkanediyl_{(C≤8)};
or a linker of the formula:

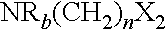

wherein:
   R_{b} is hydrogen, alkyl_{(C≤6)}, or substituted alkyl_{(C≤6)};
   n is 1, 2, or 3; and
   X₂ is arenediyl_{(C≤12)} or substituted arenediyl_{(C≤12)};
R₁ is a haloaryl_{(C≤12)}; aralkyl_{(C≤12)}; substituted aralkyl_{(C≤12)}; heterocycloalkyl_{(C≤12)}; substituted heterocycloalkyl_{(C≤12)}; 2-pyridinyl; 3-aminophenyl;
or a pharmaceutically acceptable salt thereof.

The polymer may comprise one or more sugar repeating units. The repeating unit may have the formula:

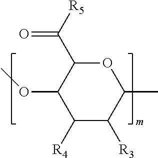

wherein:
   R₃ or R₄ are each independently hydrogen or hydroxy;
   R₅ is a hydroxy, alkoxy_{(C≤8)}, substituted alkoxy_{(C≤8)}, or a covalent bond to the linker; and
   m is a number of repeating units with a molecular weight from about 50,000 Daltons to about 500,000 Daltons.

The polymer may comprise repeating units of the formula:

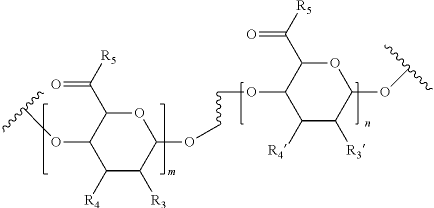

wherein:
   R₃, R₃′, R₄, or R₄, are each independently hydrogen or hydroxy;
   R₅ is a hydroxy, alkoxy_{(C≤8)}, substituted alkoxy_{(C≤8)}, or a covalent bond to the linker;
   R₅′ is a covalent bond to the linker; and
   m and n result in a number of repeating units with a molecular weight from about 50,000 Daltons to about 500,000 Daltons.

The polymer may be an acrylate polymer, such as a methacrylate polymer.

In particular, the following molecules are useful in the disclosed assays:

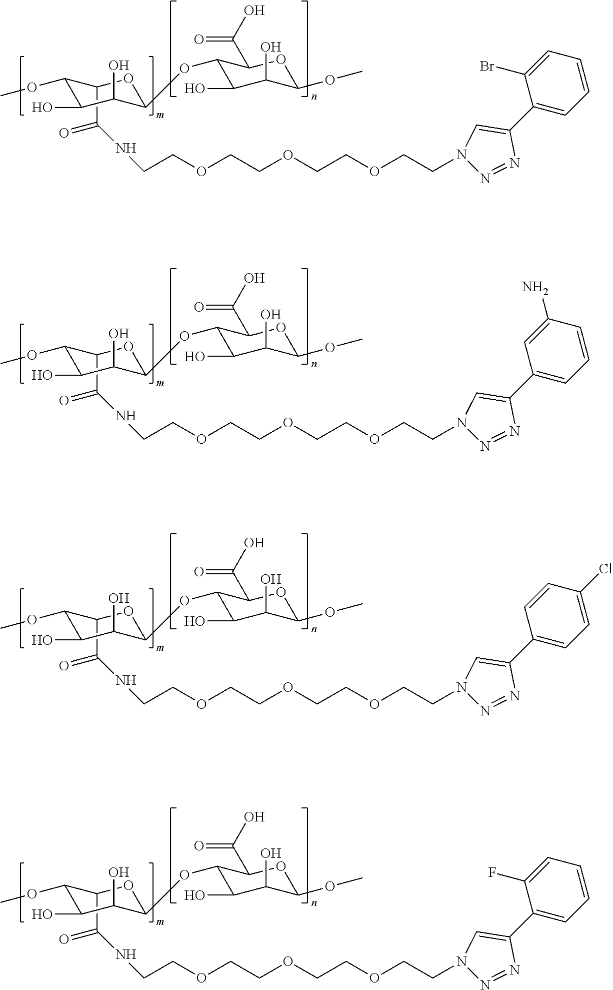

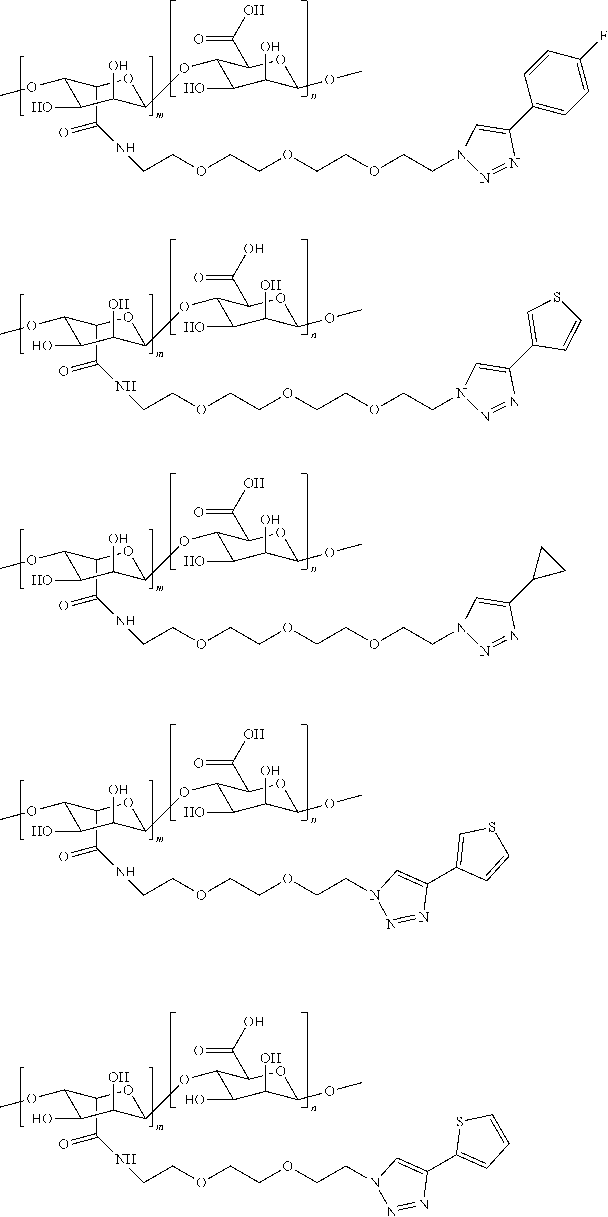

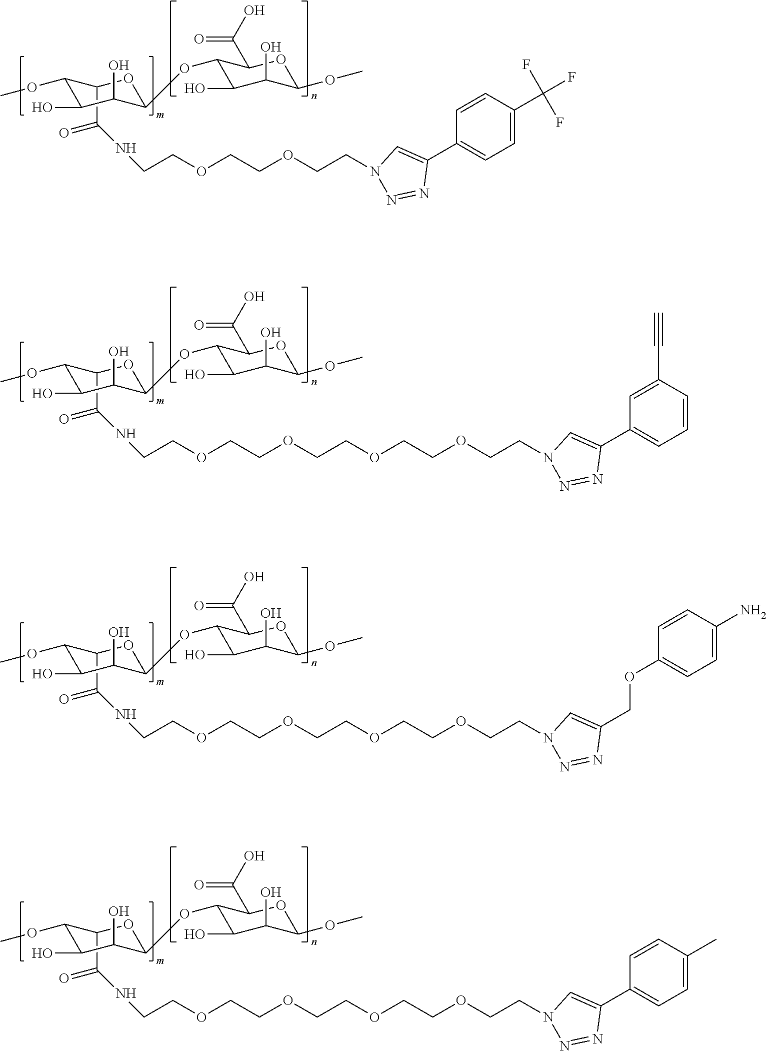

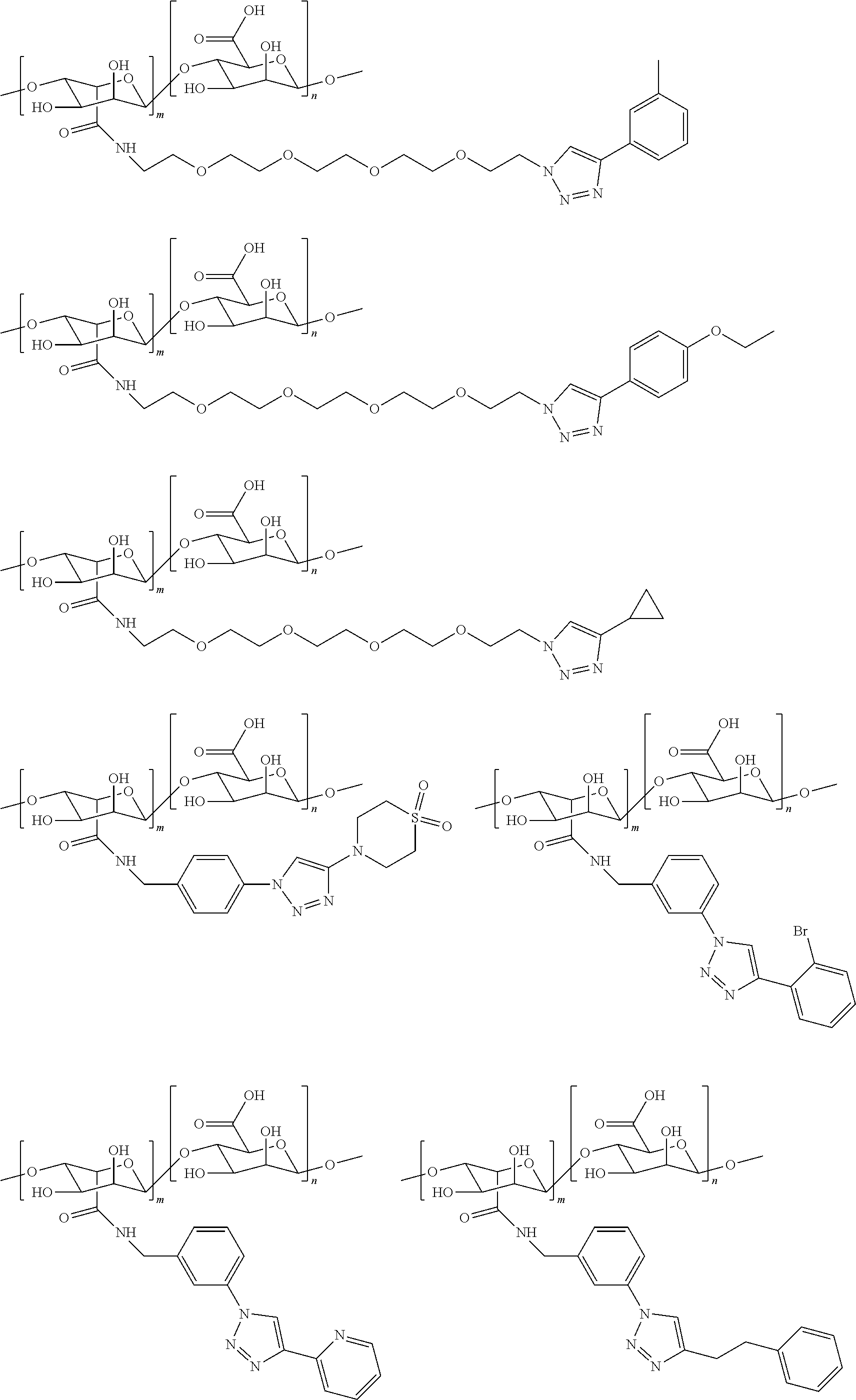

The methods may employ human or non-human animal model hosts. The material/surface can be implanted subcutaneously, intramuscularly or intraperitoneally, implanted into the brain or other organ, or inserted into a body orifice such as mouth, urethra or rectum The implanted/inserted material/surface may be left in situ. for about 24 hours or more, such as 1 day, 2 days, 3, days, 4, days, 5, days, 6, days, 7 days, 10 days, two weeks, three weeks or four weeks.

### D. CELLS

Implantable constructs described herein may contain a cell, for example, an engineered cell. A cell may be derived from any mammalian organ or tissue, including the brain, nerves, ganglia, spine, eye, heart, liver, kidney, lung, spleen, bone, thymus, lymphatic system, skin, muscle, pancreas, stomach, intestine, blood, ovary, uterus, or testes.

A cell may be derived from a donor (e.g., an allogeneic cell), derived from a subject (e.g., an autologous cell), or from another species (e.g., a xenogeneic cell). In an embodiment, a cell can be grown in cell culture, or prepared from an established cell culture line, or derived from a donor (e.g., a living donor or a cadaver). In an embodiment, a cell is genetically engineered. In another embodiment, a cell is not genetically engineered. A cell may include a stem cell, such as a reprogrammed stem cell, or an induced pluripotent cell. Exemplary cells include mesenchymal stem cells (MSCs), fibroblasts (e.g., primary fibroblasts). HEK cells (e.g., HEK293T), Jurkat cells, HeLa cells, retinal pigment epithelial (RPE) cells, HUVEC cells, NIH3T3 cells, CHO-K1 cells, COS-1 cells, COS-7 cells, PC-3 cells, HCT 116 cells, A549MCF-7 cells, HuH-7 cells, U-2 OS cells, HepG2 cells, Neuro-2a cells, and SF9 cells. In an embodiment, a cell for use in an implantable construct is an RPE cell.

A cell included in an implantable construct may produce or secrete a therapeutic agent. In an embodiment, a cell included in an implantable construct may produce or secrete a single type of therapeutic agent or a plurality of therapeutic agents. In an embodiment, an implantable construct may comprise a cell that is transduced or transfected with a nucleic acid (e.g., a vector) comprising an expression sequence of a therapeutic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the therapeutic agent and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG. Exemplary activators include the TET1 catalytic domain, P300 core, VPR, rTETR, Cas9 (e.g., from S. pyogenes or S. aureus), and Cpf1 (e.g., from L. bacterium).

An implantable construct described herein may comprise a cell or a plurality of cells. In the case of a plurality of cells, the concentration and total cell number may be varied depending on a number of factors, such as cell type, implantation location, and expected lifetime of the implantable construct. In an embodiment, the total number of cells included in an implantable construct is greater than about 2, 4, 6, 8, 10, 20, 30, 40, 50, 75, 100, 200, 250, 500, 750, 1000, 1500, 2000, 5000, 10000, or more. In an embodiment, the total number of cells included in an implantable construct is greater than about 1.0×10², 1.0×10³, 1.0×10⁴, 1.0×10⁵, 1.0×10⁶, 1.0×10⁷, 1.0×10⁸, 1.0×10⁹, 1.0×10¹⁰, or more. In an embodiment, the total number of cells included in an implantable construct is less than about than about 10000, 5000, 2500, 2000, 1500, 1000, 750, 500, 250, 200, 100, 75, 50, 40, 30, 20, 10, 8, 6, 4, 2, or less. In an embodiment, the total number of cells included in an implantable construct is less than about 1.0×10¹⁰, 1.0×10⁹, 1.0×10⁸, 1.0×10⁷, 1.0×10⁶, 1.0×10⁵, 1.0×10⁴, 1.0×10³, 1.0×10², or less. In an embodiment, a plurality of cells is present as an aggregate. In an embodiment, a plurality of cells is present as a cell dispersion.

Specific features of a cell contained within an implantable construct may be determined, e.g., prior to and/or after incorporation into the implantable construct. For example, cell viability, cell density, or cell expression level may be assessed. In an embodiment, cell viability, cell density, and cell expression level may be determined using standard techniques, such as cell microscopy, fluorescence microscopy, histology, or biochemical assay.

### E. ENGINEERED CELLS

In an embodiment, the implantable construct comprises a cell or a plurality of cells that are genetically engineered to produce or secrete a therapeutic agent. In an embodiment, the implantable construct comprises a cell producing or secreting a protein. The protein may be of any size, e.g., greater than about 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein produced or secreted by a cell may be modified, for example, by glycosylation, methylation, or other known natural or synthetic protein modification. A protein may be produced or secreted as a pre-protein or in an inactive form and may require further modification to convert it into an active form.

Proteins produced or secreted by a cell may include antibodies or antibody fragments, for example, an Fc region or variable region of an antibody. Exemplary antibodies include anti-PD-1, anti-PD-L1, anti-CTLA4, anti-TNFα, and anti-VEGF antibodies. An antibody may be monoclonal or polyclonal. Other exemplary proteins include a lipoprotein, an adhesion protein, blood clotting factor (e.g., Factor VII, Factor VIII, Factor IX, GCG, or VWF), hemoglobin, enzymes, proenkephalin, a growth factor (e.g., EGF, IGF-1, VEGF alpha, HGF, TGF beta, bFGF), or a cytokine.

A protein produced or secreted by a cell may include a hormone. Exemplary hormones include growth hormone, growth hormone releasing hormone, prolactin, lutenizing hormone (LH), anti-diuretic hormone (ADH), oxytocin, thyroid stimulating hormone (TSH), thyrotropin-release hormone (TRH), adrenocorticotropic hormone (ACTH), follicle-stimulating hormone (FSH), thyroxine, calcitonin, parathyroid hormone, aldosterone, cortisol, epinephrine, glucagon, insulin, estrogen, progesterone, and testosterone.

A protein produced or secreted by a cell may include a cytokine. A cytokine may be a pro-inflammatory cytokine or an anti-inflammatory cytokine. Example of cytokines include IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-16, IL-17, G-CSF, GM-CSF, IL-20, IFN-α, IFN-β, IFN-7, CD154, LT-β, CD70, CD153, CD178, TRAIL, TNF-α, TNF-β, SCF, M-CSF, MSP, 4-1BBL, LIF, OSM, and others. For example, a cytokine may include any cytokine described in M. J. Cameron and D. J. Kelvin, Cytokines, Chemokines, and Their Receptors (2013), Landes Biosciences, which is incorporated herein by reference in its entirety.

An implantable construct may comprise a cell expressing a single type of therapeutic agent, e.g., a single protein or nucleic acid, or may express more than one type of therapeutic agent, e.g., a plurality of proteins or nucleic acids. In an embodiment, an implantable construct comprises a cell expressing two types of therapeutic agents (e.g., two types of proteins or nucleic acids). In an embodiment, an implantable construct comprises a cell expressing three types of therapeutic agents (e.g., three types of proteins or nucleic acids). In an embodiment, an implantable construct comprises a cell expressing four types of therapeutic agents (e.g., four types of proteins or nucleic acids).

In an embodiment, an implantable construct comprises a cell expressing a single type of nucleic acid (e.g., DNA or RNA) or may express more than one type of nucleic acid, e.g., a plurality of nucleic acid (e.g., DNA or RNA). In an embodiment, an implantable construct comprises a cell expressing two types of nucleic acids (e.g., DNA or RNA). In an embodiment, an implantable construct comprises a cell expressing three types of nucleic acids (e.g., DNA or RNA). In an embodiment, an implantable construct comprises a cell expressing four types of nucleic acids (e.g., DNA or RNA).

In an embodiment, an implantable construct comprises a cell expressing a single type of protein, or may express more than one type of protein, e.g., a plurality of proteins. In an embodiment, an implantable construct comprises a cell expressing two types of proteins. In an embodiment, an implantable construct comprises a cell expressing three types of proteins. In an embodiment, an implantable construct comprises a cell expressing four types of proteins.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes.

In an embodiment, an implantable construct comprises a cell expressing a single type of antibody or antibody fragment or may express more than one type of antibody or antibody fragment, e.g., a plurality of antibodies or antibody fragments. In an embodiment, an implantable construct comprises a cell expressing two types of antibodies or antibody fragments. In an embodiment, an implantable construct comprises a cell expressing three types of antibodies or antibody fragments. In an embodiment, an implantable construct comprises a cell expressing four types of antibodies or antibody fragments.

In an embodiment, an implantable construct comprises a cell expressing a single type of hormone, or may express more than one type of hormone, e.g., a plurality of hormones. In an embodiment, an implantable construct comprises a cell expressing two types of hormones.

In an embodiment, an implantable construct comprises a cell expressing three types of hormones. In an embodiment, an implantable construct comprises a cell expressing four types of hormones.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes.

In an embodiment, an implantable construct comprises a cell expressing a single type of cytokine or may express more than one type of cytokine, e.g., a plurality of cytokines. In an embodiment, an implantable construct comprises a cell expressing two types of cytokines. In an embodiment, an implantable construct comprises a cell expressing three types of cytokines. In an embodiment, an implantable construct comprises a cell expressing four types of cytokines.

### F. FEATURES OF IMPLANTABLE CONSTRUCTS

The implantable construct described herein may take any suitable shape or morphology. For example, an implantable construct may be a sphere, spheroid, tube, cord, string, ellipsoid, disk, cylinder, sheet, torus, cube, stadiumoid, cone, pyramid, triangle, rectangle, square, or rod. An implantable construct may comprise a curved or flat section. In an embodiment, an implantable construct may be prepared through the use of a mold, resulting in a custom shape.

The implantable construct may vary in size, depending, for example, on the use or site of implantation. For example, an implantable construct may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. In an embodiment, an implantable construct may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

In an embodiment, an implantable construct comprises a pore or opening to permit passage of an object, such as a small molecule (e.g., nutrients or waste), a protein, or a nucleic acid. For example, a pore in or on an implantable construct may be greater than 0.1 nm and less than 10 m. In an embodiment, the implantable construct comprises a pore or opening with a size range of 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.1 μm to 3 μm, 0.1 μm to 2 μm.

An implantable construct described herein may comprise a chemical modification in or on any enclosed material. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. An implantable construct may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In some embodiments, the implantable construct is chemically modified with a specific density of modifications. The specific density of chemical modifications may be described as the average number of attached chemical modifications per given area. For example, the density of a chemical modification on or in an implantable construct may be 0.01, 0.1, 0.5, 1, 5, 10, 15, 20, 50, 75, 100, 200, 400, 500, 750, 1,000, 2,500, or 5,000 chemical modifications per square μm or square mm.

An implantable construct may be formulated or configured for implantation in any organ, tissue, cell, or part of a subject. For example, the implantable construct may be implanted or disposed into the intraperitoneal space of a subject. An implantable construct may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5, cm, 10 cm, or further from a tumor or other growth in a subject. An implantable construct may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

An implantable construct may be formulated for use for any period of time. For example, an implantable construct may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. An implantable construct can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). A implantable construct can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). An implantable construct can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

### G. METHODS OF TREATMENT

Described herein are implantable constructs comprising a encapsulating an engineered cell, and related methods of use thereof. In an embodiment, the implantable constructs are used to treat a disease, e.g., as described herein.

The implantable constructs described herein may further comprise an additional pharmaceutical agent, e.g., for use in combination therapy. The additional pharmaceutical agent may be disposed in or on the implantable construct or may be produced by a cell disposed in or on the implantable construct. In an embodiment, the additional pharmaceutical agent is small molecule, a protein, a peptide, a nucleic acid, an oligosaccharide, or other agent.

In an embodiment, the additional pharmaceutical agent is an immunomodulatory agent, e.g., one or more of an activator of a costimulatory molecule, an inhibitor of an immune checkpoint molecule, or an anti-inflammatory agent. In an embodiment, the immunomodulatory agent is an inhibitor of an immune checkpoint molecule (e.g., an inhibitor of PD-1, PD-L1, LAG-3, TIM-3 or CTLA4, or any combination thereof). In some embodiments, the immunomodulatory agent is a cancer vaccine.

In some embodiments, the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFR beta. In one embodiment, the inhibitor of an immune checkpoint molecule inhibits PD-1, PD-L1, LAG-3, TIM-3 or CTLA4, or any combination thereof. Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof that binds to PD-1, PD-L1, PD-L2, CTLA4, TIM3, LAG3, VISTA, BTLA, TIGIT, LAIR1, CD73, CD160, 2B4 and/or TGFR beta, or a combination thereof. In some embodiments, the immunomodulatory agent is an anti-inflammatory agent, e.g., an anti-inflammatory agent as described herein. In an embodiment, the anti-inflammatory agent is an agent that blocks, inhibits, or reduces inflammation or signaling from an inflammatory signaling pathway. In an embodiment, the anti-inflammatory agent inhibits or reduces the activity of one or more of any of the following an immune component of the subject. In an embodiment, the anti-inflammatory agent is an IL-1 or IL-1 receptor antagonist, such as anakinra, rilonacept, or canakinumab. In an embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In an embodiment, the anti-inflammatory agent is a TNF-α antagonist, e.g., an anti-TNF-α antibody, such as infliximab (REMICADE®), golimumab (SIMPONI®), adalimumab (HUMIRA®), certolizumab pegol (CIMZIA®) or etanercept. In one embodiment, the anti-inflammatory agent is a corticosteroid, e.g., as described herein.

### H. COMPOSITIONS AND ADMINISTRATIONS OF IMPLANTABLE CONSTRUCTS

The present disclosure features pharmaceutical compositions comprising an implantable construct and an engineered cell, and optionally a pharmaceutically acceptable excipient. In some embodiments, the implantable construct is provided in an effective amount in the composition. In some embodiments, the effective amount is a therapeutically effective amount. In some embodiments, the effective amount is a prophylactically effective amount.

Compositions described herein can be prepared by any method known in the art of pharmacology. In general, such preparatory methods include the steps of bringing the implantable construct into association with a carrier and/or one or more other accessory ingredients, and then, if necessary and/or desirable, shaping and/or packaging the product into a desired single- or multi-dose unit.

Compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the implantable construct may be generally equal to the dosage of the cytokine which would be administered to a subject and/or a convenient fraction of such a dosage such as, for example, one-half or one-third of such a dosage.

Relative amounts of the implantable construct, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) of any component.

The implantable construct and a pharmaceutical composition thereof may be administered or implanted orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally. In an embodiment, the implantable construct is injected subcutaneously. In an embodiment, the implantable construct is injected into the intraperitoneal space. In an embodiment, the implantable construct is injected into the intraperitoneal space. In an embodiment, the implantable constructed is delivered to the subject using a device, e.g., a cannula or catheter.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For ophthalmic use, provided compounds, compositions, and devices may be formulated as micronized suspensions or in an ointment such as petrolatum.

In an embodiment, the release of a cytokine or additional pharmaceutical agent is released in a sustained fashion. In order to prolong the effect of a particular agent, it is often desirable to slow the absorption of the agent from injection. This can be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The implantable constructs provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific therapeutic agent employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

An effective amount of a therapeutic agent released from the implantable construct may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of therapeutic agent per unit dosage form (e.g, per implantable construct).

The therapeutic agent administered may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### ENUMERATED EMBODIMENTS

1. A method of treating tissue damage and/or inducing tissue regeneration in a subject, wherein the method comprises:
   providing an implantable construct comprising an encapsulated engineered cell expressing a cytokine; and
   administering the implantable construct to the subject,
   thereby treating tissue damage and/or inducing tissue regeneration in the subject.
2. The method of embodiment 1, wherein said cytokine is an anti-inflammatory cytokine or a pro-inflammatory cytokine.
3. The method of any one of the preceding embodiments, wherein the cytokine comprises IL-10 (e.g., the cytokine is IL-10).
4. The method of any one of the preceding embodiments, wherein the implantable construct is degradable.
5. The method of any one of the preceding embodiments, wherein the implantable construct comprises a polymer.
6. The method of embodiment 5, wherein the polymer is a naturally occurring polymer or a synthetic polymer.
7. The method of any one of embodiments 5-6, wherein the polymer is a polysaccharide (e.g., alginate).
8. The method of any one of the preceding embodiments, wherein the implantable construct further comprises a triazole compound.
9. The method of any one of the preceding embodiments, wherein the tissue damage or tissue regeneration occurs in the heart tissue or lung tissue of the subject.
10. The method of any one of the preceding embodiments, wherein the heart tissue in the subject has been damaged by ischemia, such as due to coronary heart disease and/or myocardial infarction.
11 The method of embodiment 9, wherein the lung tissue has been damaged by infection, such as by viral infection.
12. The method of any one of the preceding embodiments, wherein administration comprises administration directly to the tissue (e.g., by subcutaneous injection).
13. The method of embodiment 12, wherein tissue is heart tissue or lung tissue.
14. The method of embodiment 13, wherein the tissue is damaged.
15. The method of any one of the preceding embodiments, wherein the engineered cell is an epithelial cell.
16. The method of any one of the preceding embodiments, wherein the engineered cell is selected from Chinese hamster ovary (CHO) cell, retinal pigment epithelial (ARPE-19) cell, human mammary epithelial (MCF-10a and MCF-7) cell, human embryonic kidney (HEK) cell, a mesenchymal stem cell (MSC), human umbilical vein endothelial cell (HUVEC), NIH/3T3 cell, BJ fibroblast, and human renal mix epithelial cell (HREC).
17. The method of any one of the preceding embodiments, wherein the engineered cell such as wherein said cell is engineered for regulatable expression of said cytokine.
18. The method of any one of the preceding embodiments, wherein the implantable construct is formulated as a pharmaceutical composition.
19. The method of any one of the preceding embodiments, wherein the subject is a mammal (e.g., a human).
20. A method of treating tissue damage in the heart or lung in a subject, wherein the method comprises:
   providing an implantable construct comprising an encapsulated engineered retinal pigmented epithelial (RPE) cell expressing a cytokine; and
   administering the implantable construct to the subject,
   thereby treating tissue damage in the heart or lung in the subject.
21. A method of treating a cardiovascular disease in a subject, wherein the method comprises:
   providing an implantable construct comprising an encapsulated engineered retinal pigmented epithelial (RPE) cell expressing a cytokine; and
   administering the implantable construct to the subject,
   thereby treating a cardiovascular disease in the subject.
22. A method of treating a pulmonary disease in a subject, wherein the method comprises:
   providing an implantable construct comprising an encapsulated engineered retinal pigmented epithelial (RPE) cell expressing a cytokine; and
   administering the implantable construct to the subject,
   thereby treating a pulmonary disease in the subject.

### I. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods for Heart and Lung Tissue Repair

Ischemic heart disease is the leading cause of death in the industrialized world. Heart failure (HF) develops in 20-30% of patients after myocardial infarction (MI) due to extensive scarring, which is exacerbated by a persistent increase of inflammatory macrophages that propagates injury. Cytokines, such as Interleukin-10 (IL-10), and cytokine inhibitors, such as Interleukin-1 receptor antagonists (IL-1Ra), are potent immune modulators that can mitigate inflammation, reduce infarct size, and improve ventricular function in animal models of MI. However, poor biodistribution, toxicity, infections due to systemic immunosuppression, and paradoxical pro-inflammatory responses with sustained administration represent critical challenges that prevent clinical translation of systemic cytokine therapy. In post-MI patients with HF, sustained therapeutic delivery will likely be required for months to sufficiently promote tissue repair and yield functional recovery.

Further, the severe acute respiratory syndrome coronavirus 2 (SARS-CoV-2) global pandemic has resulted in unprecedented morbidity, mortality, and economic impact. As SARS-CoV-2 is the 3rd coronavirus to cause severe global human disease in the past 2 decades, urgent solutions are required to mitigate coronavirus acute lung injury (ALI) and prevent acute respiratory distress syndrome (ARDS). Immunomodulatory therapy represents a therapeutic option to correct this imbalance in the host immune response, reduce ALI, and prevent ARDS.

The inventors report a new technique for tissue repair following heart or lung damage. The inventors' approach utilizes polymer encapsulated cells that have been engineered to continuously produce natural interleukin-10 (IL-10) to elicit a pro-regeneration immune response for applications in tissue and wound healing, tissue repair, and organ transplantation.

The inventors' objective is to develop novel and transformative immunomodulatory cell therapies to treat immune system failures that arise from MI and SARS-CoV-2 ALI/ARDS. Their approach combines hydrogels with cells engineered to produce IL-10 and IL-1Ra with tunable pharmacokinetics for local targeted delivery to the heart and lung,

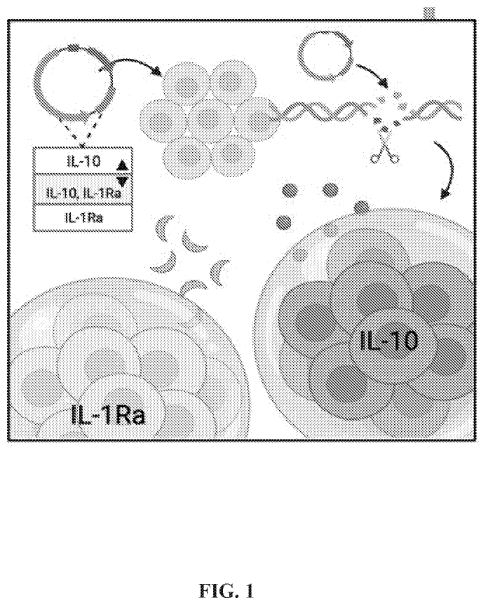

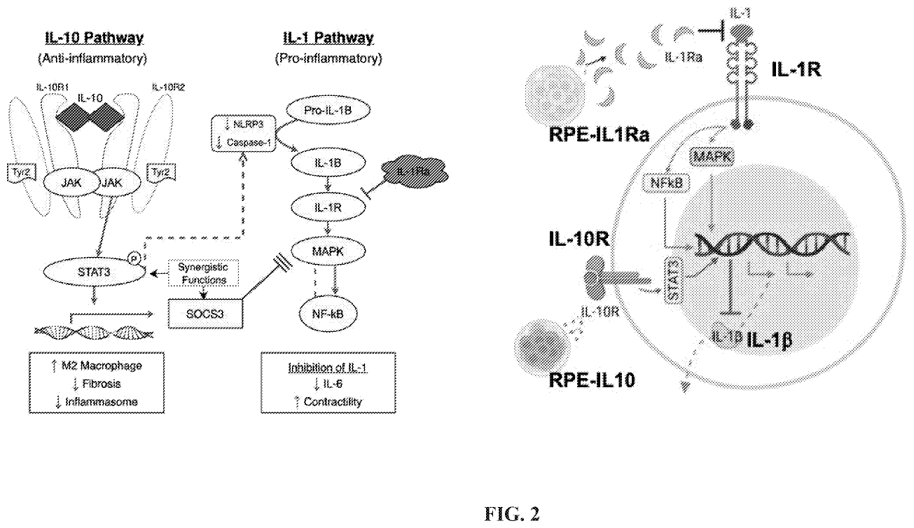

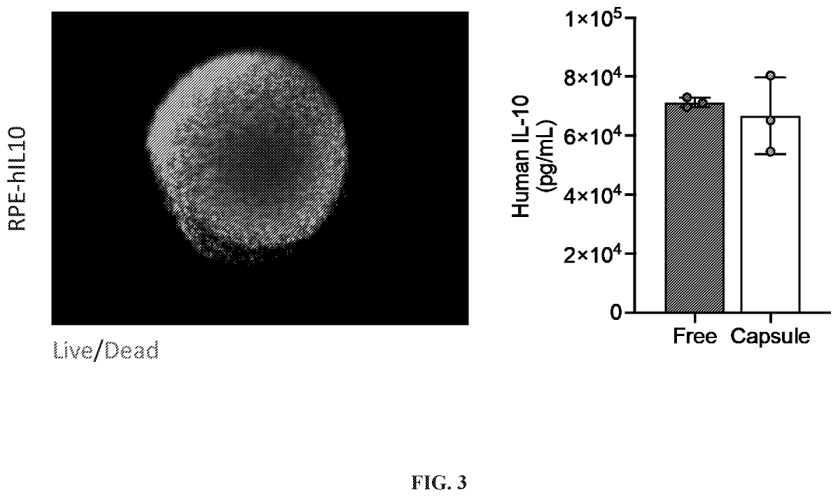

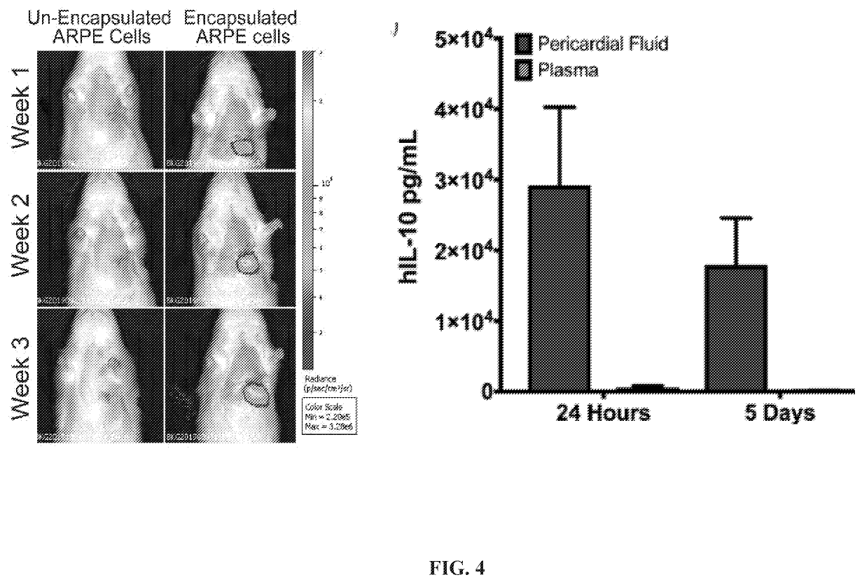

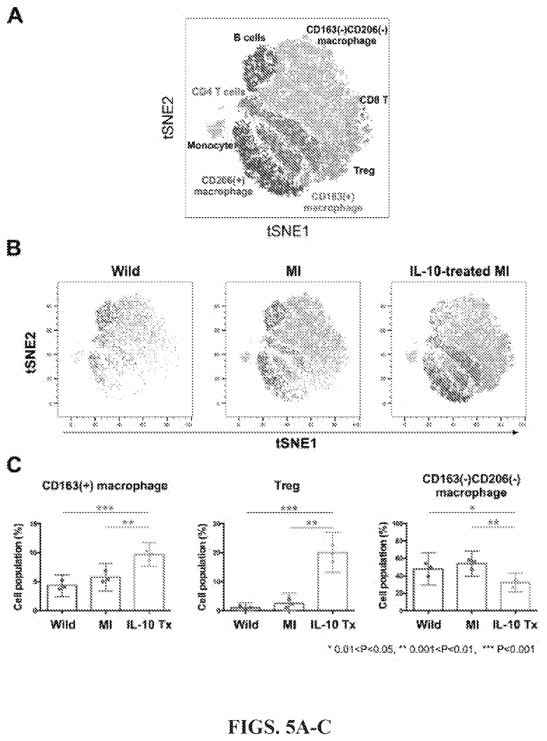

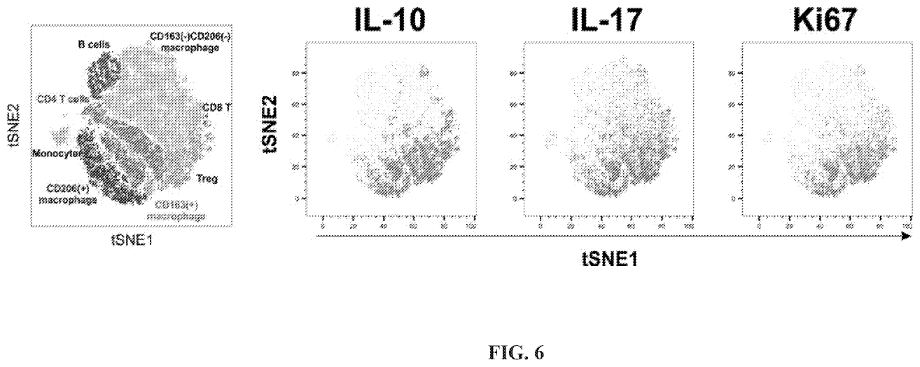

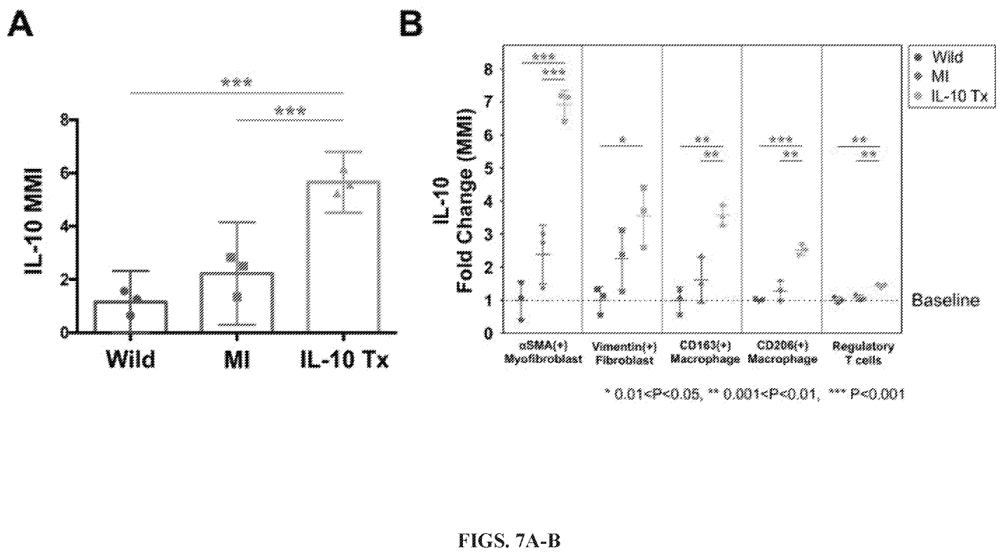

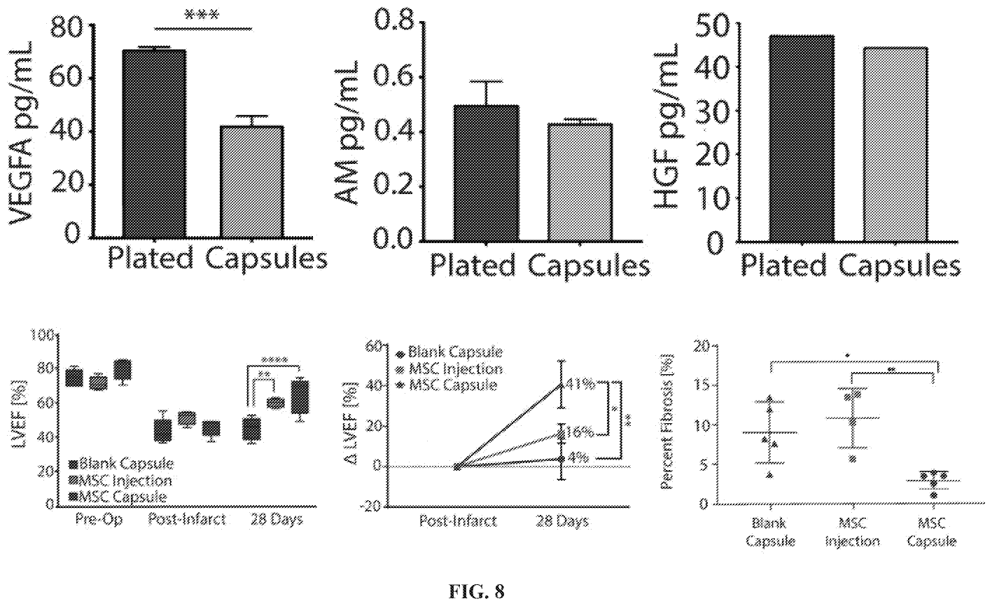

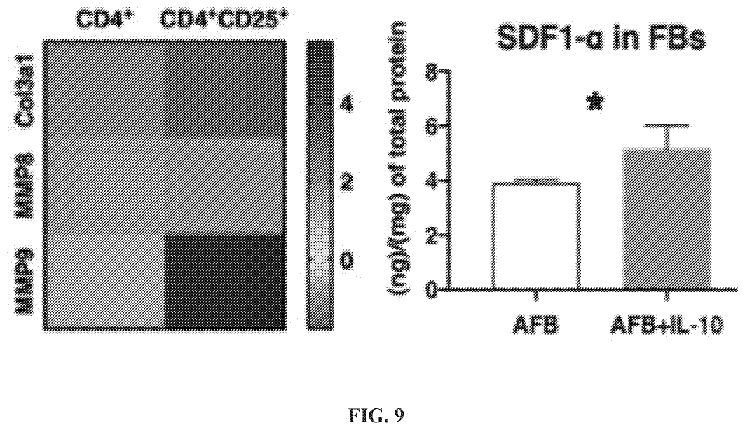

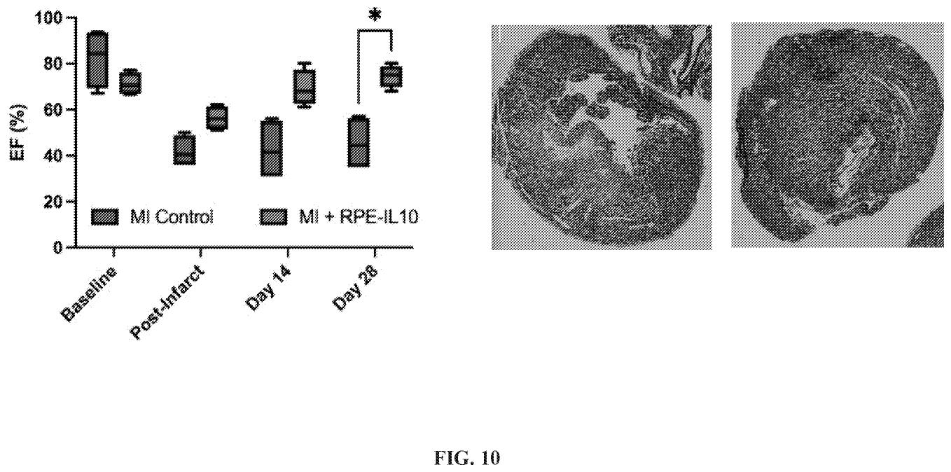

Here the inventors describe a hydrogel-based delivery system composed of cells engineered to make anti-inflammatory cytokines and agonists that repress activated immune cells in vivo. The engineered cells and the hydrogel-based spheres play an important role in modulating responses from the immune system and, when combined, allows for fine-tuned control of immunomodulatory activities. FIGS. 1-10 are illustrative of the contemplated systems and methods.

Cytokines: Cell engineering and production of cytokines has already been accomplished by the inventors. Cytokines are cell signaling proteins that are made by various cells in the body in response to specific stimuli. These proteins function to regulate communication and activation states of the cells of the immune system. The inventors have designed a cell engineering platform which used synthetic biology principles to create genetically modified cell lines that continuously produce defined concentrations of these proteins inside animals. There are three main classes of cytokines which include pro-inflammatory cytokines which function to activate immune cells, anti-inflammatory cytokines which function to repress immune cells and chemokines which function to initiate immune cell migration. The molecules in these three classes of proteins are similar in size and molecular structure and can thus be each generated with very little change in engineering technique.

The following cytokines can be produced by the inventors' engineered cells: IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-a, IFN-b, IFN-c, TNF-a, TNF-b, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17. Control over which cytokines produced by these engineered cells allows for precise coordination of the immune system. To ensure future clinical translatability, the mouse and human version of IL-10 and IL-1RA cell lines used in this project were created using the appropriate gene sequences from the NCBI. Variation to this system requires only gene of interest swapping and allows for quick and easy “plug and play” cell engineering.

Choice of base cell lines include Chinese hamster ovary (CHO), retinal pigment epithelial (ARPE-19), human mammary epithelial (MCF-10a and MCF-7), Human embryonic kidney (HEK), mesenchymal stem cells (MSC), Human umbilical vein endothelial cells (HUVEC), NIH/3T3 cells, BJ fibroblasts, Human renal mix epithelial cells (HREC).

Hydrogel: Different types of alginate can be used. These hydrogel spheres are fabricated using a custom-built, two-fluid co-axial electrostatic spraying device. The device consisted of a voltage generator that was attached to the tip of a co-axial needle and grounded to a 1:4 barium chloride:mannitol crosslinking bath. The co-axial needle was fed by two separate syringes containing 1.4% alginate solutions diluted in 0.9% saline.

The inventors can fine-tune the cytokine dose according to necessary constraints on a per patient basis by altering the number of cells in an individual sphere and/or altering the number of spheres administered per dose. For example, individual spheres can be filled with 10,000-80,000 cells. Spheres may range in size from 50 μm-3 mm.

Embodiment: Example embodiments for this technology include: 1: treatment for myocardial repair following heart attack or related tissue damage through delivery IL-10, IL-1Ra, and any combination of cytokine and/or immunosuppressive drugs. IL-10 and cytokine inhibitors, such as Interleukin-1 receptor antagonists (IL-1Ra), are potent immune modulators that can mitigate inflammation, reduce infarct size, and improve ventricular function. 2: treatment for lung repair following SARS-CoV-2 ALI/ARDS or related tissue damage through delivery IL-10 and any combination of cytokine and/or immunosuppressive drugs. 3: organ transplantation: IL-10 secreting spheres can be administered along with transplanted organs to prevent immune system activation and subsequent initiation of Graft-vs-Host disease leading to inflammation and organ rejection.

### Example 2—Methods for Treating Pleural Inflammation

Inflammation of the pleura is only one example among various conditions where excessive immune response leads to health problems. In this sense, anti-inflammatory cytokines have significant therapeutic potential for pleural inflammation and other autoimmune conditions by playing immunosuppressive roles. However, long-term immunosuppression is associated with susceptibility to malignancies and infection. Managing the timing and dosage of cytokine delivery can hasten clinical transition.

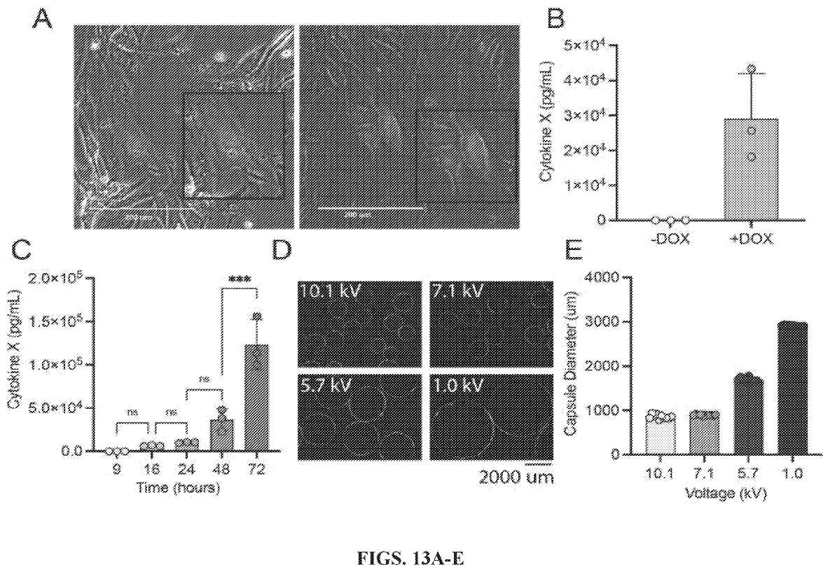

Here, the inventors investigate small molecule inducible, cell-based delivery of cytokines to retinal pigment epithelium (RPE). Using VectorBuilder®, the inventors engineer RPE cells with a construct containing a tetracycline-response element (TRE), a cytokine gene of interest, and neomycin as a selectable marker. The vector is delivered to RPE cells using Lipofectamine® 3000 (ThermoFisher). Engineered cells are selected with neomycin and the selected cells are then expanded. Doxycycline hydrochloride (DOX) treatment (2 μg/ml) induces the Tet-On system in the cells to deliver the cytokine of interest. An ELISA with media from DOX-treated cells evaluates cytokine production. FIGS. 13A-E show that cells can be externally regulated to modulate production of anti-inflammatory cytokines. This allows time and dosage specific production of anti-inflammatory cytokines, reduces vulnerabilities associated with long-term immunosuppression (e.g., infections, malignancies), and is translatable for treatment of various inflammatory and autoimmune conditions. The inventors contemplated encapsulating the cells in hydrogels for improved delivery and cell survival.

### Example 3—In Vivo Validation of Implantable Constructs Comprising Cytokine-Containing Cells

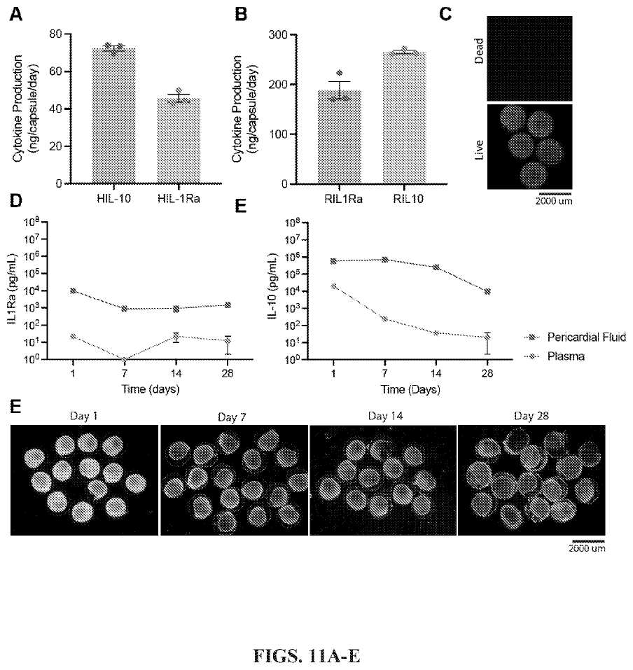

In this experiment, implantable constructs were prepared that contain cells capable of producing anti-inflammatory cytokines, namely Rat interleukin 1 receptor antagonist (RIL1Ra), Rat interleukin 10 (RIL10), Human interleukin 1 receptor antagonist (HIL1Ra), and Human interleukin 10 (HIL10). The implantable constructs were first evaluated to show that they were capable of continuously generating therapeutic cytokines (FIGS. 11A-B). Notably, FIG. 11C shows that over 90% of the encapsulated cells maintained viability, indicating their potential for long-term functionality. These implantable constructs were then implanted in the pleural space in a mouse model, it was observed that local cytokine concentrations were elevated, while systemic circulation concentrations were significantly reduced by up to 100 times at various time points (1-, 3-, 7-, and 28-days post-implant, FIGS. 11D-E). This localized effect suggests that the encapsulated cells could enable targeted cytokine delivery, minimizing potential systemic side effects. Further, the fibrosis-mitigating capability of anti-inflammatory cytokine-producing capsules for up to 28 days was investigated, as seen in darkfield microscopy images of explanted capsules (FIG. 11F). This indicates that the encapsulated cells may effectively suppress fibrosis, which could contribute to the prolonged therapeutic efficacy of the anti-inflammatory cytokine treatment.

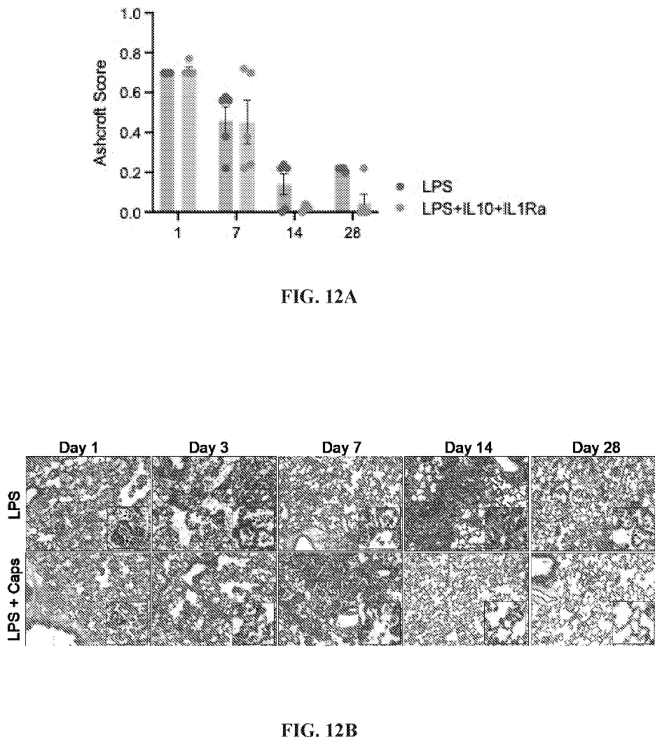

These studies were also carried out in lung tissue, where the implantable constructs were implanted in rats treat in the absence or presence of LPS and the locally produced cytokines IL-10 and IL1Ra from the implantable constructs. Histological scores from rats treated with LPS alone or LPS plus therapeutic are shown in FIGS. 12A-B, revealing that the treatment group exhibited improved lung histology compared to the LPS-alone group by day 14 (FIG. 12B). The presence of immune cell infiltration over time suggests that the encapsulated cells may have the potential to modulate the immune response in the lung tissue, contributing to the observed therapeutic effect.

Taken together, these studies show that the implantable constructs capable of producing anti-inflammatory cytokine described herein have great potential for providing a therapeutic outcome in various inflammatory conditions.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed:

1. A method of treating tissue damage and/or inducing tissue regeneration in a subject, wherein the method comprises:
providing an implantable construct comprising an encapsulated engineered cell expressing a cytokine; and
administering the implantable construct to the subject,
thereby treating tissue damage and/or inducing tissue regeneration in the subject.

2. The method of claim 1, wherein said cytokine is an anti-inflammatory cytokine or a pro-inflammatory cytokine.

3. The method of claim 1, wherein the cytokine comprises IL-10 (e.g., the cytokine is IL-10).

4. The method of claim 1, wherein the implantable construct is degradable.

5. The method of claim 1, wherein the implantable construct comprises a polymer.

6. The method of claim 5, wherein the polymer is a naturally occurring polymer or a synthetic polymer.

7. The method of any one of claims 4-5, wherein the polymer is a polysaccharide (e.g., alginate).

8. The method of claim 1, wherein the implantable construct further comprises a triazole compound.

9. The method of claim 1, wherein the tissue damage or tissue regeneration occurs in the heart tissue or lung tissue of the subject.

10. The method of claim 9, wherein the heart tissue in the subject has been damaged by ischemia, such as due to coronary heart disease and/or myocardial infarction.

11. The method of claim 9, wherein the lung tissue has been damaged by infection, such as by viral infection.

12. The method of claim 1, wherein administration comprises administration directly to the tissue (e.g., by subcutaneous injection).

13. The method of claim 12, wherein tissue is heart tissue or lung tissue.

14. The method of claim 13, wherein the tissue is damaged.

15. The method of claim 1, wherein the engineered cell is an epithelial cell.

16. The method of claim 1, wherein the engineered cell is selected from Chinese hamster ovary (CHO) cell, retinal pigment epithelial (ARPE-19) cell, human mammary epithelial (MCF-10a and MCF-7) cell, human embryonic kidney (HEK) cell, a mesenchymal stem cell (MSC), human umbilical vein endothelial cell (HUVEC), NIH/3T3 cell, BJ fibroblast, and human renal mix epithelial cell (HREC).

17. The method of claim 1, wherein the engineered cell such as wherein said cell is engineered for regulatable expression of said cytokine.

18. The method of claim 1, wherein the implantable construct is formulated as a pharmaceutical composition.

19. The method of claim 1, wherein the subject is a mammal (e.g., a human).

20. A method of treating tissue damage in the heart or lung in a subject, wherein the method comprises:
providing an implantable construct comprising an encapsulated engineered retinal pigmented epithelial (RPE) cell expressing a cytokine; and
administering the implantable construct to the subject,
thereby treating tissue damage in the heart or lung in the subject.

21. A method of treating a cardiovascular disease in a subject, wherein the method comprises:
providing an implantable construct comprising an encapsulated engineered retinal pigmented epithelial (RPE) cell expressing a cytokine; and
administering the implantable construct to the subject,
thereby treating a cardiovascular disease in the subject.

22. A method of treating a pulmonary disease in a subject, wherein the method comprises:
providing an implantable construct comprising an encapsulated engineered retinal pigmented epithelial (RPE) cell expressing a cytokine; and
administering the implantable construct to the subject,
thereby treating a pulmonary disease in the subject.
